(19) United States
(12) Patent Application Publication
(10) Pub. No.: US 2026/0306267 A1
(43) Pub. Date: Oct. 8, 2026

(54) A CUSTOMIZED ACETABULAR CAGE WITH INTEGRATED AUGMENT

(71) Applicant: Ketan JAJAL, Vallabh Vidyanagar, Gujarat (IN)

(72) Inventors: Ketan JAJAL, Vallabh Vidyanagar, Gujarat (IN); Prashant CHHATWANI, Vadodara, Gujarat (IN); Navdeep DORIYA, Surendranagar, Gujarat (IN)

(21) Appl. No.: 19/483,326

(22) PCT Filed: May 11, 2024
(86) PCT No.: PCT/IN2024/050520

§ 371 Date: Nov. 11, 2025

(30) Foreign Application Priority Data
May 11, 2023 (IN) 202321026699

(51) Int. Cl. A61F 2/34; A61F 2/30; A61F 2/46

(52) U.S. Cl. CPC A61F 2/34; A61F 2/30734; A61F 2/30767; A61F 2/30942; A61F 2/4609; A61F 2002/30578; A61F 2002/30736; A61F 2002/3093; A61F 2002/30948; A61F 2002/30985

(57) ABSTRACT

The present invention relates to a customized acetabular cage. More particularly, the present invention relates to a hip or acetabular cage which has integrated with it the bone augment required for a particular hip arthroplasty surgery. The augment is integrated into the acetabular cage in a seamless manner. The present hip cage is further provided with flanges having minimum dimensions which reduce the requirement for incision. The seamlessly integrated bone augment reduces the overall size of the implant and also ensures a perfect fit.

### FIELD OF INVENTION

The present invention relates to the field of hip cage implant that includes a patient-specific acetabular cage 1 with integrated augment 2. More particularly, the present invention relates to a hip cage implant includes a patient-specific acetabular cage 1 and other side of acetabular as dome-shaped surface integrated by a periphery bone augment 2.

### BACKGROUND OF INVENTION

The hip is one of the body's largest joints. It is a ball-and-socket joint. The socket is formed by the acetabulum, which is part of the pelvis bone. The ball is the femoral head, which is the upper end of the femur (thighbone). The bone surfaces of the ball and socket are covered with articular cartilage, a smooth tissue that cushions the ends of the bones and enables them to move easily. A thin tissue called the synovial membrane surrounds the hip joint. In a healthy hip, this membrane makes a small amount of fluid that lubricates the cartilage and eliminates almost all friction during hip movement. Bands of tissue called ligaments (the hip capsule) connect the ball to the socket and provide stability to the joint.

In a total hip replacement (also called total hip arthroplasty), the damaged bone and cartilage is removed and replaced with prosthetic components.
The damaged femoral head is removed and replaced with a metal stem that is placed into the hollow center of the femur. The femoral stem may be either cemented or “press fit” into the bone.
A metal or ceramic ball is placed on the upper part of the stem. This ball replaces the damaged femoral head that was removed.
The damaged cartilage surface of the socket (acetabulum) is removed and replaced with a metal socket. Screws or cement are sometimes used to hold the socket in place.
A plastic, ceramic, or metal spacer is inserted between the new ball and the socket to allow for a smooth gliding surface.

The present invention relates to providing a socket of acetabulum which is customized as specifically for the subject patient.

Impaction bone grafting and acetabular cementation is one of the ways in which acetabular defects were dealt with. Impaction bone grafting and acetabular cementation has been described for treating segmental or combined acetabular defects. In this technique, the acetabulum is reamed to bleeding bone, and segmental defects are reconstructed using metal mesh or solid graft. Femoral head autograft or fresh-frozen allograft is morselized into <1 cm diameter cancellous pieces. The graft is impacted, and bone cement is pressurized throughout the graft prior to implantation of an all-polyethylene cup.

Despite reasonable outcomes using cemented techniques, cemented reconstruction are not recommended as the first line treatment since these techniques are labour intensive and do not provide biologic fixation. Additionally, allograft reconstruction carries a risk of graft resorption and infection.

Further, another technique implemented is the ring and cage reconstruction. Rings and cages historically have been reserved for cases of acetabular revision with inadequate bone stock where a “bridge” is required to span the defect. Rings and cages are secured to the ilium and ischium with screws placed through small flanges, and a liner is then cemented into the appropriate version and abduction.

Ring and cage reconstruction provides a relatively low-cost method for bridging acetabular defects that cannot be achieved by a hemispheric cup alone. The major disadvantage of ring and cage reconstruction is the risk of subsequent implant failure, especially if used in “higher demand” patient populations. This technique also relies upon cementation rather than complete biologic fixation, which may compromise long-term component longevity.

Another method is the method of structural allograft cement. In the case of significant acetabular bone loss or developmental hip dysplasia (where the superolateral acetabulum may be absent), reconstruction can be achieved using structural allograft alongside acetabular cementation. In this technique, allograft bone (e.g. femoral head) is shaped to fill the acetabular defect and fixed in place using cancellous screws, and the acetabular cup is cemented into place.

Sternheim et al. retrospectively studied 18 cemented acetabular cups and 27 uncemented acetabular cups with an average of 18-year follow-up. 18 Ten and 20-year cup survival, respectively, was only 67% and 36% for cemented cups compared to 88% and 76% for uncemented cups, respectively. Therefore, structural allograft with cementation may be associated with greater loosening compared to use of structural allograft with a hemispheric implant alone and generally is not recommended by the authors given the availability of other more durable reconstruction methods.

Uncemented hemispheric acetabular reconstruction is another technique. Although the acetabular reconstruction techniques described in sections above all involved cemented constructs, modern hip reconstruction commonly employs cementless, biologic fixation strategies. Cementless acetabular reconstruction with a hemispheric implant is the universally recommended treatment for Paprosky type 1 and 2 defects. 12 Excellent mid-to long-term outcomes of uncemented hemispheric acetabular reconstruction have been shown in several studies, with reports of >90% survivorship when using aseptic loosening as an endpoint.

The main advantages of uncemented hemispheric acetabular reconstruction include a more familiar technique for surgeons and the potential for biologic fixation. However, uncemented hemispheric reconstruction techniques do require good pre-existing bone stock and at least 50% implant contact with host bone.

Uncemented structural allograft reconstruction is another method of hip reconstruction. Hemispheric shells can also be used in the setting of inadequate column support with more extensive bone loss (e.g. Paprosky types 2 and 3) e in conjunction with bulk structural allograft e according to the severity of acetabular bone loss. Femoral head allograft was used in type 2B defects for reconstruction of the superolateral pillar. Distal femur and proximal tibia allograft may be needed for reconstruction of type 3A acetabular defects.

Despite the favourable survivability of structural allografts in revision THA, porous metal augments have grown increasingly popular as an alternative reconstruction strategy for achieving biologic fixation and avoiding graft resorption over time. However, given that structural allografts may enhance future bone stock.

Bone augmentation for dental implants are sometimes necessary when a patient does not have enough bone available to properly anchor the screw or other implant hardware. Porous metal modular augments have revolutionized the treatment of severe acetabular bone loss over the past one and a half decades. Augments secured with screws and implanted prior to placement of the acetabular shell provide primary stability (e.g. anterosuperior, posteroinferior columns) for the overall acetabular construct and are more than just simple bone void “fillers.” Augments may also be placed posterosuperiorly following cup insertion (e.g. in the case of inadequate purchase of screws through the cup), thereby providing supplemental fixation. Augments should be thought of as extensions of the acetabular shell and are “unitized” to the cup using cement in order to minimize metal debris from micro-motion during the early post-operative period and extend the surface area of contact for the overall construct.

Porous metal augments have the advantage of allowing for intra-operative customization of the acetabular construct based on the bone loss defect. However, greater technical expertise and understanding of augment function is required for successful use of these devices for complex acetabular defects.

Cup-cage reconstruction has been recently popularized as a hybrid technique using a porous metal acetabular shell in conjunction with an acetabular cage. This construct is reserved for treatment of severe type 3 acetabular defects with or without a chronic pelvic discontinuity. In this technique, a porous metal shell is implanted and secured with as many screws as possible; this may be supplemented by one or more modular porous metal augments. An acetabular cage is then implanted over the cup and spans the ilium and ischium. Screws are placed through the cage (and sequentially the cup, when possible) into host bone.

The major advantage of cup-cage constructs is that compared to traditional ring and cage reconstructions, they allow for biologic fixation. However, there is still a risk for cage fracture over time. There is also a tendency to place the cup too vertical in order to accommodate the cage, and placement of the ischial flange can prove difficult in the setting of severe ischial osteolysis.

The present invention is a customized multiflange acetabular components (CMACs) are customized implants that are created through detailed preoperative planning. A three-dimensional CT scan is used to create a 3D-printed acrylic model of the affected hemipelvis. This model incorporates the planned hip center and designates screw placement, length, and trajectory. Once the model structure is agreed upon between the manufacturer and surgeon, a custom porous or hydroxyapatite-coated tri-flanged titanium replica is created. The CMAC has the capacity to span acetabular defects in a more rigid fashion and therefore can be used to achieve bony union even in the setting of pelvic discontinuity.

The major advantage of CMACs is the theoretical power to perfectly match distorted host anatomy with significant bone loss. However, CMACs require approximately 6 weeks of lead time, and they rarely fit perfectly according to the pre-operative plan due to additional bone loss encountered during component removal. CMACs also have a higher rate of post-operative instability, which the authors speculate may be related to superior gluteal nerve denervation that can occur when the abductors are elevated off the outer table during placement of the ilial flange.

The present invention aims to overcome the disadvantages of the methods of the prior art by providing an acetabular cage with a perfect fit augment for every patient which reduces the risk of aseptic loosening and implant failure. The present acetabular cage has minimalistic flange design which reduce the required incision. A porous lattice structure is provided at the contact surfaces for the enhanced bone regeneration. The positioning of the screws is customized to maximize bon purchase. Multiple bead cavities are provided for better cement mantal anchorage. The design of the enables its usage with any cemented implant liner.

### SUMMARY OF THE INVENTION

One of the aspect of the present invention relates to the field of hip cage implant 100 that includes a patient-specific acetabular cage 1 with integrated augment 2. More particularly, the present invention relates to a hip cage implant includes a patient-specific acetabular cage 1 wherein the other side of acetabular is a rear convex surface 1b (dome-shaped) surface integrated by a periphery bone augment 2. The dome-shaped surface is a three-dimensional patient-specific surface designed to anatomically match and be received into an acetabulum of the patient, based on a three-dimensional image of a hip joint of the patient reconstructed from a medical scan of the hip joint. The hip cage implant includes microporous structures a plurality of custom directed locking screw position with maximum bone purchase and augment 2 having minimalistic flanges design of custom hip cage requiring smaller incision and having patient-specific shapes and orientations for guiding corresponding osteotomies relative to the acetabulum for correcting hip dysplasia.

In some embodiments, the hip cage implant that includes a patient-specific acetabular cage 1 with integrated augment 2 include an one or more annular flange 3 extending from the periphery of the dome-shaped surface of acetabular cage 1. The augment 2 has a patient-specific surface engageable to a periacetabular area of the acetabulum of the patient. A plurality of elongated slots are defined by the flange and oriented around the periphery in a patient-specific orientation for guiding a cutting instrument to re-orient the acetabulum relative to the pelvis.

Another aspect of the present invention relates to customized hip cage implant 100 comprising: a customized acetabular cage 1 with integrated augment 2;. an outer side of acetabular cage 1 is a rear convex surface 1b (dome-shaped) surface having microporous structures 6, an inner surface, an opening acetabular cage 1; one or more annular flange 3 extending from the periphery of the dome-shaped surface of acetabular cage 1; a plurality of custom directed fixing screws comprises (a) locking Screws 8 (b) countersink dome screws 9 and (c) countersink compression screws 10 with hydroxyapatite stimulates the osteoinduction process and the structure of the surrounding bone tissue becomes significantly denser; wherein the dome-shaped surface is a three-dimensional patient-specific surface designed to anatomically match and be received into an acetabulum of the patient.

Another aspect of the present invention relates to customized hip cage implant 100 that includes a patient-specific acetabular cage 1 with integrated augment 2 is customized for the patient which reduces the risk of aseptic loosening and implant comprises (a) Locking Screws is pre-defined such that the screw passes through the cortical bone, cancellous bone and then again cortical bone allowing bi-cortical purchase of bone to have stable fixation of the implant with bone and to avoid micromotion between bone and implant; (b) Countersink Dome Screws are the non-locking Countersink acetabulum screws which are used to fix acetabular cage 1 with cortical and cancellous bone of the pelvis; (c) Countersink Compression Screws are the non-locking Countersink acetabulum screws which are used to fix acetabular cage 1 with bi-cortical bones on the iliac region to have compressive effect between the bone and implant surfaces. The process of designing of the custom hip cage implant comprising (a) Locking Screws (b) Countersink Dome Screws and (c) Countersink Compression Screws allows for the said hip cage implant to have integrated bone augment eliminating the requirement of loose pieces of bone augments thus reducing the risk of post operation implant failure.

Another aspect of the present invention relates to customized hip cage implant also provide an implant for a femoral head of a patient. The implant includes a acetabular cage 1 on the femoral head of the patient and correcting a defect of the femoral head. The acetabular cage 1 with integrated augment 2 is designed preoperatively to have a patient-specific first surface for articulation with the patient's acetabulum and a patient-specific periphery mateable with a periphery of the defect. The acetabular cage 1 and the surface of the cage is continuous to a remaining healthy surface of the femoral head. The defect can be a bone defect, a cartilage defect or a combination thereof.

Another aspect of the present invention relates to customized hip cage implant also provide an threaded hole at center of cup of acetabular cage 1 to fix impactor handle during placement in the surgery. The method includes engaging an acetabulum of a pelvis of a patient with a patient-specific hip cage implant which is guide by impactor handle and positioning a patient-specific acetabular cage 1 with integrated augment 2 which is a dome-shaped to its outer surface into a complementary surface of the acetabulum. The method also includes guiding a cutting instrument through a first elongated slot of a flange surrounding the dome-shaped surface, performing a first osteotomy through the first elongated slot, and correcting an orientation of the acetabulum.

Another aspect of the present invention relates to hip cage implant includes threaded hole at centre of cup of acetabular cage 1 to fix impactor handle 14 during placement and positioning of hip cage implant during the surgery.

Another aspect of the present invention relates to hip cage implant includes has hook between ischium and pubis bone.

Further areas of applicability of the present teachings will become apparent from the description provided hereinafter. It should be understood that the description and specific examples are intended for purposes of illustration only and are not intended to limit the scope of the present teachings.

The customized design of the hip cage implant or acetabular cage allows for the flanges to have minimalistic dimensions which minimises the incision sizes. The flange and screw positions are customized for each case. The said custom screw positioning maximises bone purchase.

Another aspect of the present invention is the porous lattice structures at the contact surfaces which enhance bone regeneration.

Yet another aspect of the present invention is to provide strengthening ridges on the convex surface of the cage along lines where the curvature of the surface is changing.

### BRIEF DESCRIPTION OF DRAWINGS

The drawings constitute a part of this invention and include exemplary embodiments of the present invention illustrating various objects and features thereof.

FIG. 1: Illustration of the acetabular cage with an integrated augment with the concave surface in the view.

FIG. 2: Illustration of the acetabular cage with an integrated augment with the convex surface in the view.

FIG. 3: Illustration of the acetabular cage with an integrated augment implanted on a hip bone.

FIG. 4: Illustration of the mirroring of the pelvis.

FIG. 5: Illustration of generation of the best fit acetabular cage 1.

FIG. 6: Illustration of the creation of the plane and the centre at the acetabulum centre.

FIG. 7: Illustration of acetabular cage 1 at the centre of the acetabulum.

FIG. 8: Illustration of the thickening of the plane along the edge of the acetabular cage 1.

FIG. 9: Illustration of fixation of the screws inside the best fit acetabular cage 1.

FIG. 10: Illustration of the screws in the flanges.

FIG. 11: Illustration of the creation of augment.

FIG. 12: Illustration of housing of the screws.

FIG. 13: Illustration of the perforations for the screws in the cage.

FIG. 14: Illustration of the Boolean operation between the cage and the pelvis.

FIG. 15: Illustration of the thickening of the bottom surface of the cage.

FIG. 16: Illustration of Boolean removal of the removal of thickness from the original cage.

FIG. 17: Illustration of the porous structure applied on the surface of the cage.

FIG. 18: Illustration of the creation of the ridges combined with the porous cage.

FIG. 19: Illustration of the intersection of the screw housings with the cage.

FIG. 20: Illustration of the final design of the customized acetabular cage.

FIG. 21A: Illustration of the customized acetabular cage with integrated augment and one annular flange

FIG. 21B: Illustration of the customized acetabular cage with integrated augment and two annular flange

FIG. 21c: Illustration of the customized acetabular cage with integrated augment and one annular flange

FIG. 22: Illustration of the customized acetabular cage with integrated augment and Dog ears

FIG. 23: Illustration of the customized acetabular cage with integrated augment and hook

FIG. 24: Illustration of the customized acetabular cage with integrated augment with impactor handle.

FIG. 25: Illustration of the customized acetabular cage with integrated augment with impactor handle and threaded hole at center of acetabular cage 1.

FIG. 26: Illustration of the customized acetabular cage with integrated augment with (a) Locking Screws (b) Countersink Dome Screws and (c) Countersink Compression Screws.

FIG. 27: Comparison of present invention customized hip cage implant with Burch-Schneider Cage implant (Stress test).

FIG. 28: Comparison of present invention customized hip cage implant with Burch-Shnider Reinforcement Hip cage from Zimmer.

### DETAILED DESCRIPTION OF THE INVENTION

For the purpose of promoting, an understanding of the principles of the invention, references will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

Reference herein to “one embodiment” or “another embodiment” means that a particular feature, structure, or characteristics described in connection with the embodiment can be included in at least one embodiment of the invention. The appearances of the phrase “in one embodiment” in various places in a specification are not necessarily all referring to the same embodiment, nor are separate or alternative embodiments mutually exclusive of other embodiments. Further, the diagrams representing one or more embodiments of the invention do not inherently indicate any particular order nor imply any limitations in the invention.

The hip joint is a ball and socket joint wherein the acetabular cage is the socket part of the joint. The present invention relates to a hip or acetabular cage which is customized for every patient.

When the different cases of hip arthroplasty are considered, the nature and extent of the deformation of the hip joint varies from cases to case. Therefore, there is an increased probability of implant failure if a “one size fits all” approach is adopted with respect to the implants. The present invention overcomes this issue by providing an acetabular implant which is customized for each patient and the bone augment is integrated into the said hip/acetabular cage. The augment of the implant being customized as per the specific deformity of the patient and its integration into the hip cage ensures a perfect fit and thus drastically reduces the chances of implant failure.

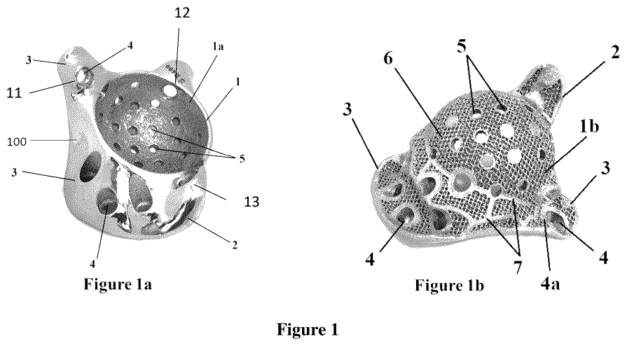

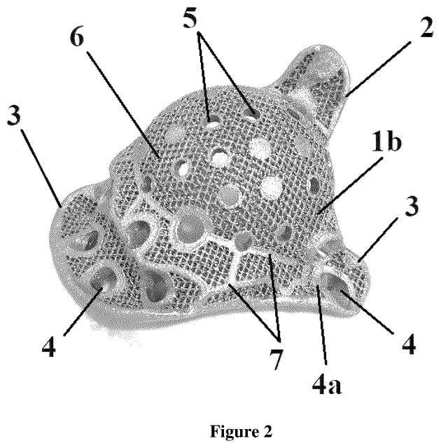

The FIG. 1 and FIG. 2 illustrate a customized acetabular cage 1 with integrated augment 2. The customized design of the said acetabular cage allows for the minimization of the dimensions of the flanges 3 through which screws are driven into the bone for the fixation of the implant. The said reduction in the size of the flanges 3 reduces the required incision.

The custom minimal flanges 3 are provided with custom screw openings 4 which are directed in a manner to maximize bone purchase, i.e., the contact of the screw threads with the bone tissue for anchorage. The flanges 3 being minimal refers to their projection from the acetabular cage being at a minimum.

Further, multiple bead cavities 5 are provided which improve cement metal anchorage. The acetabular cage of the present invention is also provided with a porous lattice micro porous structures 6 at the contact surfaces for an increased bone regeneration. The contact surface refers to the convex surface of the hip cage facing the proximal region of the pelvis.

The rear convex surface 1b of the acetabular cage 1 is provided with a plurality of ridges 7 for the increased structural strengthening of the cage against failure from compressive, crushing, bending or tensile forces. The said ridges 7 are provided especially along the line where the curvature is changing such as where the cage meets the augment part or the cage meets the flange part. The ridges 7 are regions of the rear or convex surface of the acetabular cage 1 where no porosity is provided.

In an embodiment of the present invention, the annular region 4a around the screw openings 4 is not provided with no porosity to provide a higher structural strength to the said annular region 4a. The increased structural strength of the said annular region 4a around the screw openings 4 safeguard the damaging of the said parts of the hip cage during the fixation of the screws.

The customization of the acetabular cage is a part of the pre-surgery planning and is performed in an accompanying design software.

The present teachings generally provide a patient-specific hip cage implant that includes a patient-specific acetabular cage 1 with integrated augment 2 for use in orthopedic surgery, such as in joint replacement or revision surgery, for example. The patient-specific hip cage implant components is prepared with computer-assisted image methods. Computer modeling for obtaining three dimensional images of the patient's anatomy using MRI, CT or Other medical scans of the patient's anatomy, the patient-specific prosthesis components, and the patient-specific acetabular cage 1 with integrated augment 2 and templates can be designed using various CAD programs and/or software.

The hip cage implant that includes a patient-specific acetabular cage 1 with integrated augment 2 is generally configured to match the anatomy of a specific patient. The patient-specific hip cage implant is formed using computer modeling based on the patient's 3-D anatomic image and have an engagement surface that is made to conformingly contact and match a three-dimensional image of the patient's bone surface (with or without cartilage or other soft tissue), by the computer methods discussed above.

The patient-specific hip cage implant 100 can include custom-made acetabular cage 1 with integrated augment 2, one or more annular flange extending from the periphery of the dome-shaped surface of acetabular cage 1; Custom directed locking screw holes comprising (a) Locking Screws 8 (b) Countersink Dome Screws 9 and (c) Countersink Compression Screws 10; threaded hole at center of acetabular cage 1 to fix impactor handle such as, for example, Custom directed locking screw holes 11, 12, 13 that can be used for supporting or guiding other instruments, such as drill guides or for inserting pins or other fasteners according to a surgeon-approved pre-operative plan. The patient-specific alignment hip cage implant 100 comprises acetabular cage 1, integrated augment 2 can be used in minimally invasive surgery, and in particular in surgery with multiple minimally-invasive incisions.

The medical scan data obtained can be sent to a manufacturer. The medical scan data can be used to construct a three-dimensional image of the joint and provide an initial implant fitting and alignment in a computer file form or other computer representation. The initial implant fitting and alignment can be obtained using an alignment method, such as alignment protocols used by individual surgeons.

The outcome of the initial fitting is an initial surgical plan that can be printed or provided in electronic form with corresponding viewing software. The initial surgical plan can be surgeon-specific, when using surgeon-specific alignment protocols. The initial surgical plan, in a computer file form associated with interactive software, can be sent to the surgeon, or other medical practitioner, for review. The surgeon can incrementally manipulate the position of images of implant components in an interactive image of the joint. Additionally, the surgeon can select or modify resection planes, types of implants and orientations of implant insertion. For example, the surgeon may select patient-specific ante version and abduction angles for acetabular implants, as discussed below. After the surgeon modifies and/or approves the surgical plan, the surgeon can send the final, approved plan to the manufacturer.

The acetabular cage 1 body wall provides a plurality of openings therethrough. Some of these openings are bone screw receptive openings that are reinforced with an annular reinforcement that extends away from the convex surface of the acetabular cage 1 body. Others of the openings are openings that allow cement to flow through the concave and convex layers of the acetabular cage 1 body. A threaded hole accepts a correspondingly threaded instrument that allows for ring insertion.

In a one of the embodiment hip cage implant 100 comprises acetabular cage 1, integrated augment 2 and at least one flange extends away from the rim of the acetabular cage 1 and helps attach the acetabular cage 1 body to the patient's bone. The integrated augment 2 and at least one flange helps secure the acetabular cage 1.

In a second embodiment hip cage implant 100 comprises acetabular cage 1, integrated augment 2, a plurality of radially and circumferentially spaced flanges (preferably one or two or three) extend away from the acetabular cage 1 body.

After the surgical plan is approved by the surgeon, patient-specific alignment hip cage implant 100 comprises acetabular cage 1, integrated augment 2 can be developed using a CAD program or imaging software, such as the software provided by Materialise, for example, according to the surgical plan. The hip cage implant 100 comprises acetabular cage 1, integrated augment 2 can be manufactured by various stereo lithography methods, selective laser sintering, fused deposition modeling or other rapid prototyping methods. In some embodiments, computer instructions of tool paths for machining the patient-specific alignment hip cage implant 100 comprises acetabular cage 1, integrated augment 2 can be generated and stored in a tool path data file. The tool path can be provided as input to a CNC mill or other automated machining system, and the alignment hip cage implant 100 comprises acetabular cage 1, integrated augment 2 can be machined from polymer, ceramic, metal or other suitable material, and sterilized. The sterilized alignment hip cage implant 100 comprises acetabular cage 1, integrated augment 2 can be shipped to the surgeon or medical facility, for use during the surgical procedure.

The present teachings provide a patient-specific hip cage implant 100 that includes a patient-specific acetabular cage 1 with integrated augment 2 is inserted in the acetabulum of a patient's pelvis in a guided orientation. Referring to FIGS. 1, 2, 3, 7, 13, 14, 15, hip cage implant 100 includes a patient-specific acetabular cage 1 wherein the rear convex surface 1b (dome-shaped) and integrated by a periphery bone augment 2. The rear convex surface 1b (dome-shaped surface) is a three-dimensional patient-specific surface designed to anatomically match and be received into an acetabulum of the patient, based on a three-dimensional image of a hip joint of the patient reconstructed from a medical scan of the hip joint. The hip cage implant includes microporous structures a plurality of custom directed locking screw position with maximum bone purchase and augment 2 having minimalistic flanges design of custom hip cage requiring smaller incision and having patient-specific shapes and orientations for guiding corresponding osteotomies relative to the acetabulum for correcting hip dysplasia.

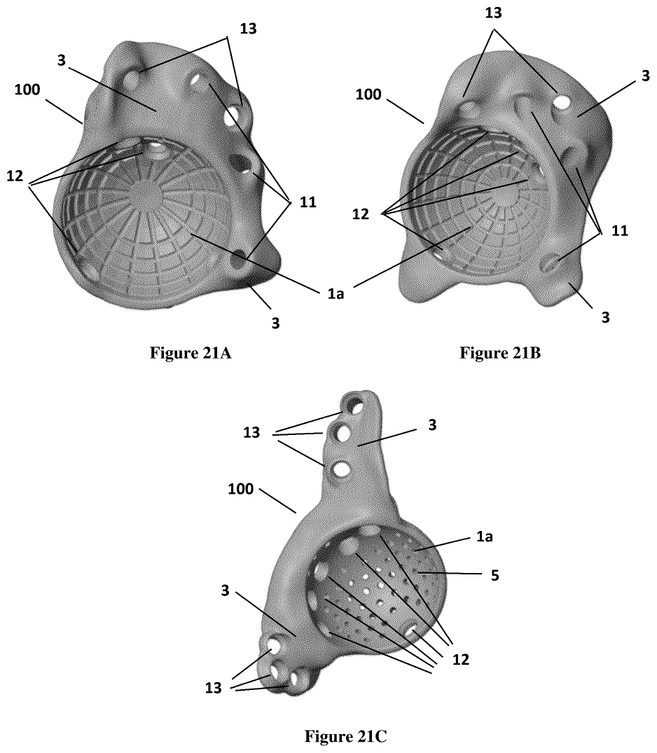

In some embodiments, the hip cage implant 100 that includes a patient-specific acetabular cage 1 with integrated augment 2 include an one or more annular flange extending from the periphery of the dome-shaped surface of acetabular cage 1. The augment 2 has a patient-specific surface engageable to a periacetabular area of the acetabulum of the patient. A plurality of elongated slots are defined by the flange and oriented around the periphery in a patient-specific orientation for guiding a cutting instrument to re-orient the acetabulum relative to the pelvis. Referring to FIGS. 21A, 21B, 21C hip cage implant 100 may have one-flange (1 flanges) or bi-flange (2 flanges) as per requirement.

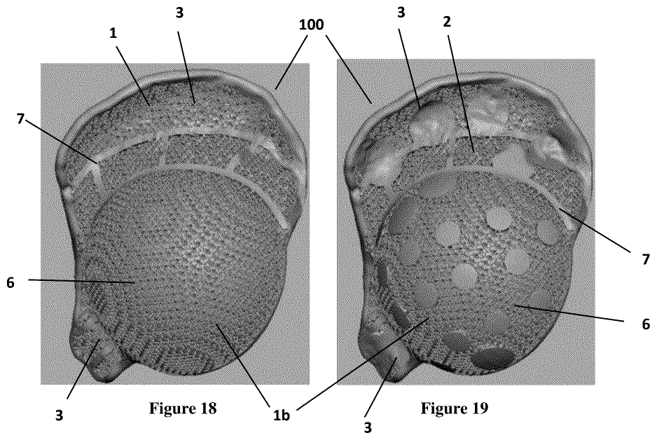

Referring to FIGS. 18, 19, an exemplary hip cage implant 100 according to the present teachings can include a acetabular cage 1 with integrated augment 2 for engaging the acetabulum. The acetabulum-engaging acetabular cage 1 can be custom-made or patient-specific to conform and mirror an acetabulum of a specific patient by using three-dimensional image of the acetabulum and surrounding pelvic area of the patient, as described above. The acetabular cage 1 rear convex surface 1b enables the nest or closely mate relative to the acetabulum of the patient.

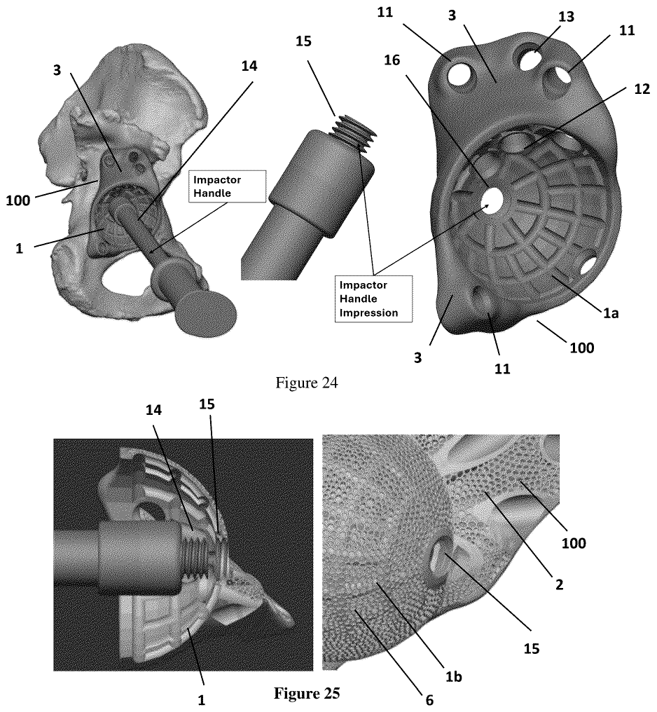

Referring to FIG. 24, 25, hip cage implant 100 also provide an threaded hole at center of acetabular cage 1 to fix impactor handle 14 during placement in the surgery. The method includes engaging an acetabulum of a pelvis of a patient with a patient-specific hip cage implant 100 which is guide by impactor handle 14 and positioning a patient-specific acetabular cage 1 with integrated augment 2 which is a dome-shaped to its outer surface into a complementary surface of the acetabulum.

In one of the embodiments, the hip cage implant 100 installation is guide by impactor handle 14 for the positioning a patient-specific acetabular cage 1 with integrated augment 2. More specifically, the impactor handle 14 include a distal end impactor handle threaded impression 15, which can be inserted and engage with threaded hole at center of cup 16. The acetabular cage 1 wherein rear convex surface 1b (dome-shaped) can be mounted on acetabulum of the patient by aligning a plurality annular flange 3 extending from the periphery of the dome-shaped surface 1b of acetabular cage 1 with corresponding cut-outs on a peripheral edge of the hip cage implant 100. The acetabular cage 1 is a three-dimensional patient-specific surface designed to anatomically match and be received into an acetabulum of the patient, based on a three-dimensional image of a hip joint of the patient reconstructed from a medical scan of the hip joint, as illustrated in FIG. 25

The hip cage implant 100 can be made of any bio compatible material, such as metal, ceramic or polymer. The hip cage implant 100 can be constructed by various manufacturing methods depending of the selected material, including, for example, 3D printing, machining, casting, molding, stereolithography or other layer deposition methods.

For certain patients, acetabular implants may be implanted after certain hip joint abnormalities are corrected, such as for example, after correcting developmental, congenital or other hip joint dysplasia or other misalignment between a femoral head and the acetabulum 20 of the pelvis 21.

Hip dysplasia is a condition typically characterized by poor coverage of the superior and anterior surface of the femoral head, a shallow acetabulum and/or short femoral neck. Various surgical procedures have been developed to correct hip dysplasia, including periacetabular osteotomy methods, single open osteotomy, double osteotomy, and triple osteotomy. Various osteotomy methods are known by the names of the surgeons who developed the corrective surgical methods.

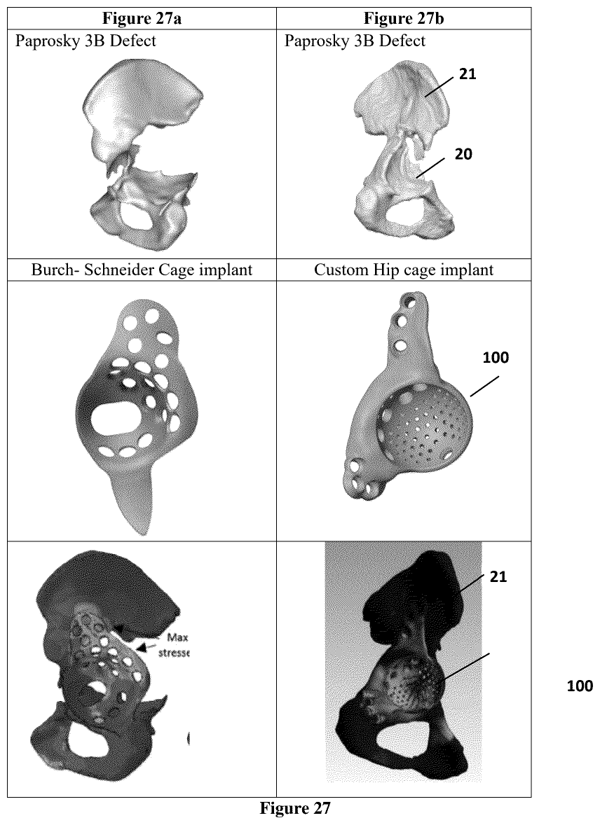

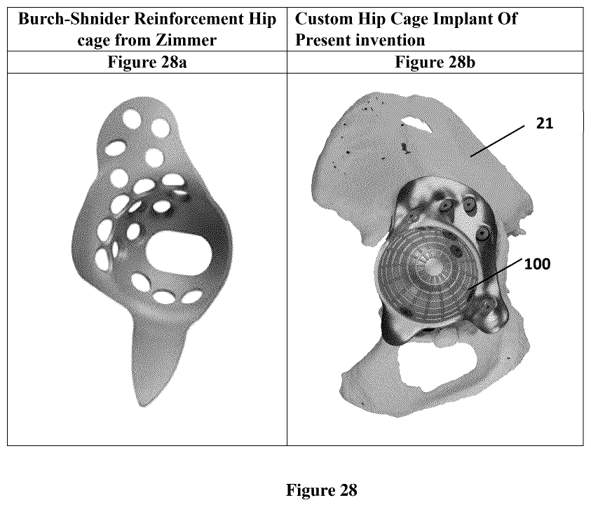

During the corrective procedure for hip dysplasia, the surgeon typically determines the location and orientation of the desired osteotomies and performs the osteotomies substantial in a free-hand manner. The present teaching provide patient-specific hip cage implant 100 comprises acetabular cage 1, integrated augment 2 with impactor handle that can assist and guide the surgeon to perform the osteotomies according to a pre-operative plan of osteotomies designed for the particular patient. Referring to FIGS. 27 and 28, an exemplary patient-specific hip cage implant 100 can be prepared according to the present teachings to perform pre-planned osteotomies and correct developmental hip dysplasia or related conditions.

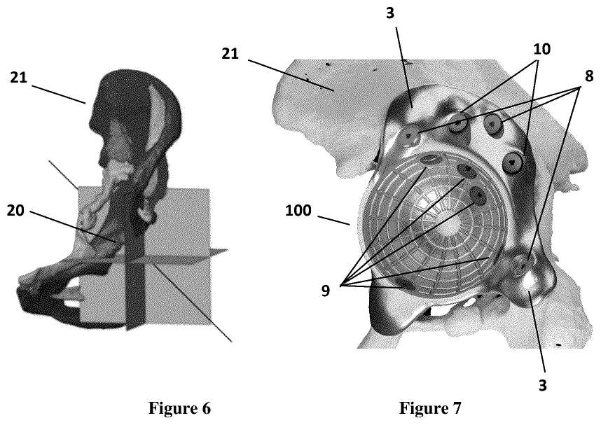

The hip cage implant 100 provides a patient-specific acetabular cage 1 with integrated augment 2 that is prepared during pre-operative planning for the surgical procedure based on a three-dimensional image of the hip joint of the patient. The three-dimensional image of the hip joint of the patient is developed by software, as discussed above, using MRI, CT, fluoroscopy, ultrasound, or other medical scans of the particular patient. Referring to FIGS. 1 and 6, the hip cage implant 100 includes a three-dimensional curved patient-specific acetabular cage 1 with integrated augment 2. The patient-specific acetabular cage 1 rear convex surface 1b is dome-shaped and has a complementary shape that matches the acetabulum 20 of the pelvis 21 of the patient for nesting therein in only one position. An annular flange 3 extends from a periphery of the rear convex surface 1b dome-shaped surface of the patient-specific acetabular cage 1. The annular flange 3 has a substantially flat or annular region or planar upper surface and an opposite lower surface that can be patient-specific at least in some or all of the areas that engage the periacetabular surface of the pelvis 21. The flange 3 of the acetabular cage 1 is designed during the pre-operative plan to define a plurality of elongated through-slots of acetabular cage 1.

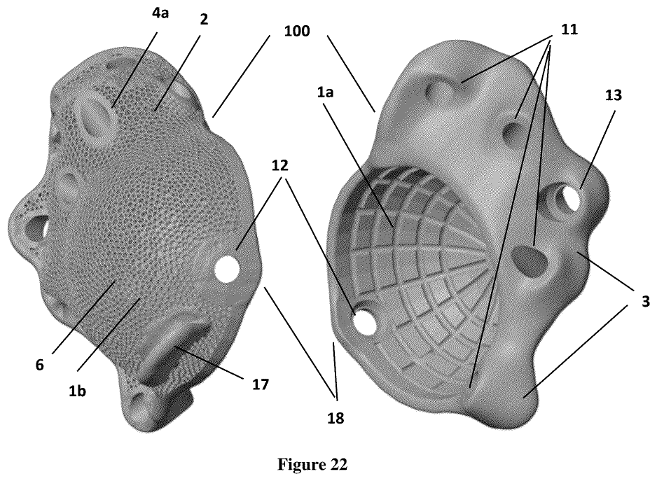

In one illustrative embodiment, patient-specific acetabular cage 1 arranged peripherally around with the dog ears 18 for custom locations for better bone purchase to resect the periacetabular area of the acetabulum 20 or the pelvis 21 for correction the hip dysplasia using a periacetabular resection procedure. As can be seen in FIG. 22, some of the elongated slots dog ears 18 may intersect such that after resection, the acetabulum can be rotated relative to the femoral head such that the orientation of the acetabulum is corrected. The orientation and location of the elongated slots dog ears 18 is patient-specific and is determined pre-operatively to match the acetabulum of the patient for enabling the acetabular resections.

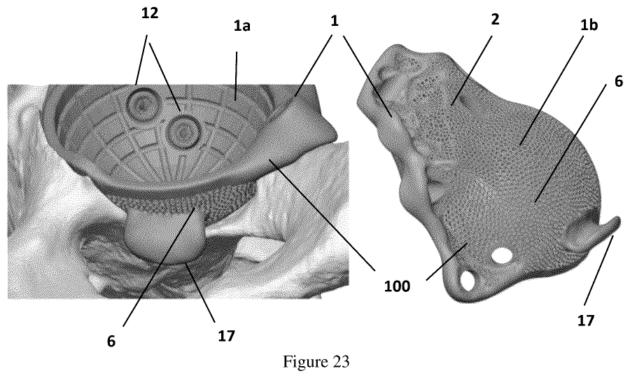

The patient-specific acetabular cage 1 can also include a straight elongated patient-specific hook 17 between ischium and pubis bone for performing the single osteotomy procedure, the acetabulum remains hinged in the ischial and pubic region at a patient-specific location determined by the uncut region that remains in the acetabulum based on the location of the osteotomy and the corresponding hook 17, illustrated in FIG. 23.

The patient-specific acetabular cage 1 can also include an arcuate or curved hook 17 that can be used optionally in other corrective procedures. The acetabular cage 1 can be provided with integrated augment 2 and annular flange 3 (one or two or three) or hook 17, all of which can be designed pre-operatively to be patient-specific and provide the surgeon with more than one option of performing the procedure based on intra-operative considerations. The various options can be provided in a single acetabular cage 1, as illustrated in FIG. 21, 22, 23, 26, 27. Alternatively, more than one acetabular cage 1 can be provided for various combinations of annular flange 3 and curved hook 17. For example, one acetabular cage 1 can include only one annular flange 3 the and curved hook 17, while another acetabular cage 1 can include one or two annular flange 3 or curved hook 17 or both the annular flange 3 and curved hook 17.

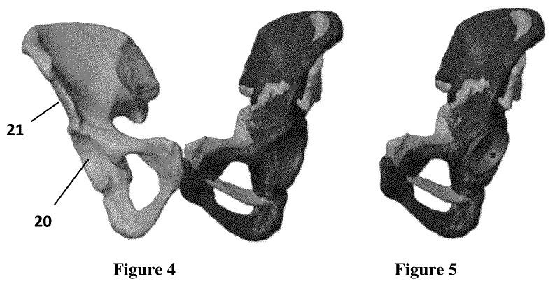

In some instances acetabular dysplasia can be corrected solely by performing one or more osteotomies and re-orienting the acetabulum, as described above, or in combination with a total or partial hip replacement, or solely by a hip replacement. Referring to FIGS. 4-11, an hip cage implant 100 can be selected for implantation during the pre-operative stage, with or without re-orientation of the acetabulum depending on the patient's condition and the surgeon's preferences. The hip cage implant 100 can be patient-specific and designed during the pre-operative stage based on the three dimensional image of the hip joint of the patient such that an outer surface microporous structures 6 of the acetabular cage 1 with integrated augment 2 matches in a complementary manner with the patient's acetabulum and other patient-specific kinematic considerations. The outer surface a microporous structure 6 on the rear convex surface 1b of the acetabular cage 1 with integrated augment 2 can be coated or otherwise covered with material promoting ingrowth, such a porous metal, including, porous titanium construct. The microporous structure 6 can include a plurality of apertures for use with fasteners to attach to the acetabulum 20 of the patient.

Other anatomic defects can also be associated with the patient's acetabulum and identified in the medical scans of the hip joint of the patient. Referring to FIG. 27, for example, a weakened or defective area can extend from a annular flange 3 of the acetabulum 20 of the patient. A patient-specific acetabular cage 1 with integrated augment 2 (henceforth collectively hip cage implant 100) can be prepared during the preoperative plan to strengthen, fill or replace and generally correct the defective area and be with the annular flange 3 and/or curved hook 17, as illustrated in FIGS. 27-28. The hip cage implant 100 include a three-dimensional rear convex surface 1b (dome-shaped) outer patient-specific Microporous structures (porous lattice structure) 6 surface to match and replace the defective area, with or without any prior milling, burring procedure, as selected by the surgeon during the pre-operative planning stage. The outer microporous structures 6 of the acetabular cage 1 with integrated augment 2 is covered with ingrowth-promoting material, such as porous titanium, and include a plurality of through apertures for fixation to the pelvis 21.

Referring to FIGS. 27-28, anatomic defects identified in the medical scan of the hip joint of the patient may also include bone defects associated with the femoral head and/or cartilage defects in the acetabulum of femoral head, or combinations thereof. Such defects can be corrected with patient-specific implants that can be designed and selected during the preoperative plan based on the three-dimensional image of the hip joint of the patient constructed from MRI or CT pr other medical scans of the patient, as discussed above.

The hip cage implant 100 can be made of any biocompatible material, including metal alloys, such as titanium alloys (Ti-6Al-4V), or cobalt-chromium alloys (Co—Cr—Mo) or ingrowth-promoting porous metal or plastic materials including Ultra High Molecular Weight Polyethylene (UHMWPE), PEEK, or other polymers.

The hip cage implant 100 installing to achieve a “press-fit” effect, the size of the hip cage implant 100 should exceed the internal size of the acetabular cage 1 after its processing with cutters by 2 mm. The insertion into the acetabulum is controlled under hammer blows on the acetabular cage 1 with integrated augment 2, taking into account the resistance of the bone tissue and the decrease in diastasis (the distance between the bottom of the acetabular cage 1 and the bone tissue) through the holes for the installation of bone screws. The tightness of the acetabular cage 1 is determined by gently rocking the hip cage implant 100. In cases where doubts remain about the strength of the primary fixation, it is advisable to strengthen the hip cage implant 100 with fixing screws made of titanium (bone screws diameter 6.5 mm), wherein fixing screws comprises (a) locking Screws 8 (b) countersink dome screws 9 and (c) countersink compression screws 10.

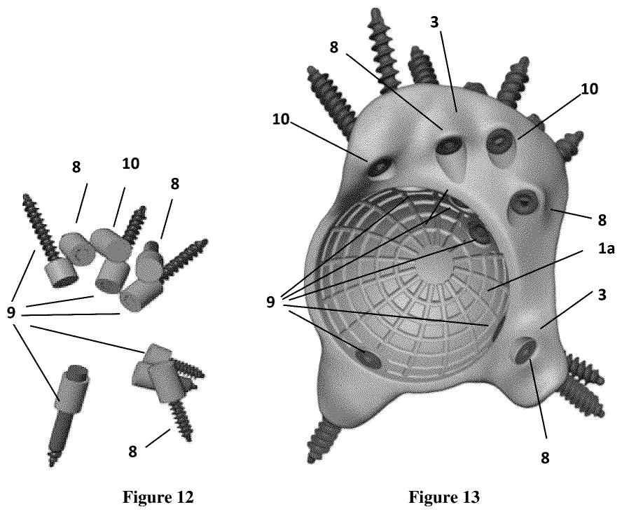

Referring to FIGS. 12-15, hip cage implant that includes a patient-specific acetabular cage 1 with integrated augment 2 is customized for the patient which reduces the risk of aseptic loosening and implant comprises (a) Locking Screws is pre-defined such that the screw passes through the cortical bone, cancellous bone and then again cortical bone allowing bi-cortical purchase of bone to have stable fixation of the implant with bone and to avoid micromotion between bone and implant; (b) Countersink Dome Screws are the non-locking Countersink acetabulum screws which are used to fix acetabular cage 1 with cortical and cancellous bone of the pelvis; (c) Countersink Compression Screws are the non-locking Countersink acetabulum screws which are used to fix acetabular cage 1 with bi-cortical bones on the iliac region to have compressive effect between the bone and implant surfaces. The process of designing of the custom hip cage implant comprising (a) Locking Screws (b) Countersink Dome Screws and (c) Countersink Compression Screws allows for the said hip cage implant to have integrated bone augment eliminating the requirement of loose pieces of bone augments thus reducing the risk of post operation implant failure

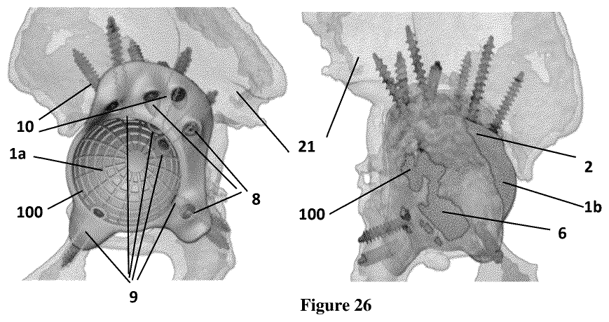

The hip cage implant 100 that includes a patient-specific acetabular cage 1 with integrated augment 2 is customized for the patient which reduces the risk of aseptic loosening and implant comprises locking Screws 8 inserted into threaded holes 11 on integrated augment 2 passing through the cortical bone, cancellous bone and then again cortical bone allowing bi-cortical purchase of bone to have stable fixation of the implant with bone and to avoid micromotion between bone and implant. It should be borne in mind that if the primary planting density depends on the correctly selected cutter size and additional fixation with screws, then the secondary fixation is provided by an active process of osseointegration—the formation of bone substance inside the microporous structures 6 of the acetabular cage 1. Already within the first month after the operation, a strong bone-implantation block is formed, which ensures a stable position of the hip cage implant 100 for a long time. Countersink Dome Screws 9 is inserted into Countersink Dome holes 12 in acetabular cage 1 are the non-locking Countersink acetabulum screws which are used to fix acetabular cage 1 with cortical and cancellous bone of the pelvis. Countersink Dome holes 12 in acetabular cage 1 for Countersink Dome Screws 9 used to fix acetabular cage 1 with cortical and cancellous bone of the pelvis. These screws provide long columnar fixation of acetabular cage 1 with respect to the bone. The length of these screws can be as high as 70 mm allowing the screw to hold the acetabular cage 1 relatively to a very strong bone available in the pelvic region. The countersink compression Screws 10 is inserted into countersink compression holes 13 on integrated augment 2 are the non-locking countersink acetabulum screws which are used to fix acetabular cage 1 with bi-cortical bones on the iliac region to have compressive effect between the bone and implant surfaces, which will result proper bone contact to promote bone in-growth in the porous region of the microporous structures 6 rear convex surface 1b of acetabular cage 1 and integrated augment 2, as illustrated in FIG. 26.

The titanium hip cage implant 100 comprising fixing screws (a) Locking Screws 8 (b) Countersink Dome Screws 9 and (c) Countersink Compression Screws 10 with hydroxyapatite stimulates the osteoinduction process and the structure of the surrounding bone tissue becomes significantly denser. The latter is of great importance for the prevention of loosening of the elements of the endoprosthesis in severe osteoporosis. The osteoinduction process is also important when filling small bone defects, for example, persistent diastasis (1-1.5 mm) between the acetabular cage 1 and the bone bed of the cavity.

The present teachings provide various patient-specific instruments, including alignment guides acetabular cage 1 with integrated augment 2 and annular flange 3 and/or elongated hook 17 and/or dog ears 18 for hip joint arthroplasty. The present teachings also provide patient-specific implants for the acetabulum and femoral head for correcting bone and/or cartilage defects.

The foregoing discussion discloses and describes merely exemplary arrangements of the present teachings. Furthermore, the mixing and matching of features, elements and/or functions between various embodiments is expressly contemplated herein, so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the teachings of the invention without departing from the essential scope thereof. One skilled in the art will readily recognize from such discussion, and from the accompanying drawings and claims, that various changes, modifications and variations can be made therein without departing from the spirit and scope of the present teachings as defined in the following claims.

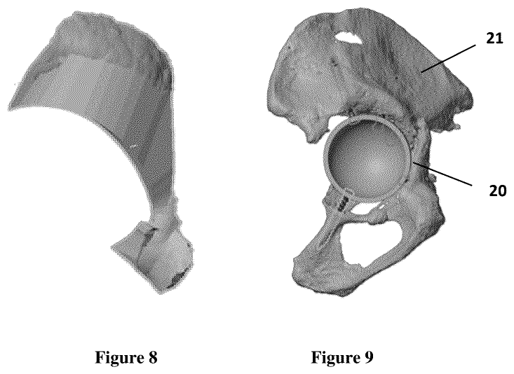

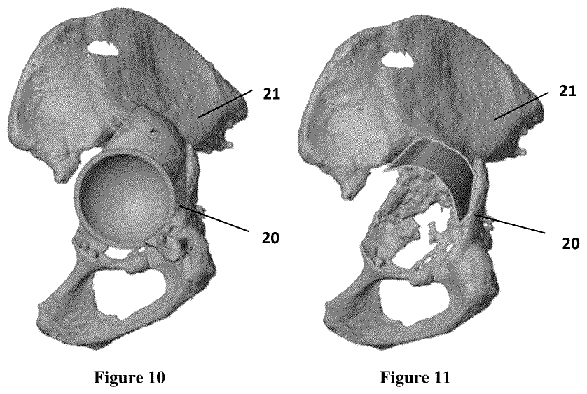

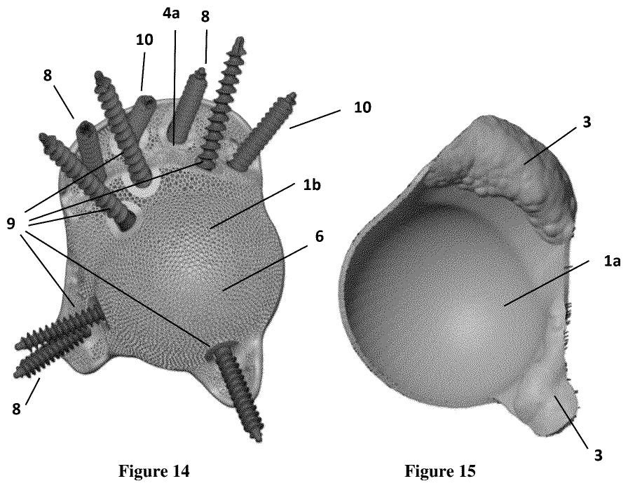

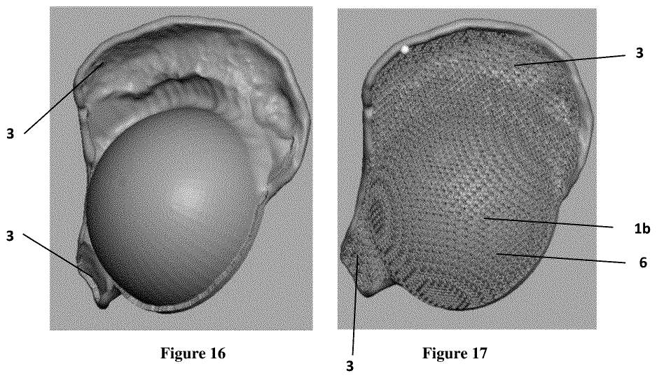

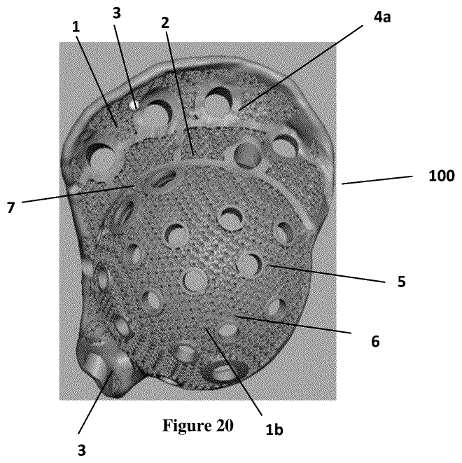

The method for the customization of the acetabular cage of the present invention, for executing the revision hip arthroplasty, comprises the following steps:
1. Creating a radiographic image of the hip region of the patient preferably by CT scanning and feeding the said radiographic image into the design software.
2. Mirroring of the healthy side of the pelvic bone to match with the deformed side of the bone within the design software. The FIG. 4 illustrates the said process of mirroring of the healthy side of the bone to fill the deformed parts of the deformed side of the bone.
3. Using the mirrored healthy side the best fit acetabular cage 1 for the deformed side and the acetabulum centre is generated. As illustrated in the FIG. 5, the best fit acetabular cage 1 is used to generate the acetabulum centre. The distal side pelvic is matched and the position of the cup of acetabular cage 1 is changed if required.
4. At the acetabulum centre a plane is created as illustrated in the FIG. 6 and also a acetabular cage 1 axis is drawn as shown in the said figure.
5. With the drawn acetabular cage 1 axis as the reference and by considering the best fit acetabular cage 1 and the acetabulum centre the acetabular cage 1 is decided as illustrated in the FIG. 7.
6. A plane is created along the outer edge of the acetabular cage 1 and joined to the patient specific surface and then then is thickened by 3 mm. The same has been illustrated in the FIG. 8.
7. Fixation screws are placed inside the best fit cup of acetabular cage 1, as shown in the FIG. 9, and the maximum depth achieved by the said screws in the bones is checked.
8. Flange fixation screws are placed on top of the flange, as illustrated in the FIG. 10. Appropriate depth of the said screw inside the bone is ensured and the fouling of a screw with any other screw is avoided.
9. Augment is created on the bottom side of the flange as shown in the FIG. 11.
10. Housings are created for all the screws and perforations are created on the acetabular cage 1, as illustrated in the FIG. 12, and their proper positions are verified as in FIG. 13.
11. Boolean operation is performed between the cage and the pelvis and the corners are smoothened, which has been illustrated in the FIG. 14.
12. The bottom surface of the cage, as shown in the FIG. 15, is selected and is thickened by 1.5 mm.
13. Boolean operation is performed to remove 1.5 mm of surface from the 3 mm original cage as shown in the FIG. 16.
14. DC8S3 porous structure pattern is applied on a 1.5 mm surface and then is combined into the original cage as illustrated in the FIG. 17.
15. Ridges are created on the original cage and intersected and then the said intersected ridges are combined with the porous cage as shown in the FIG. 18.
16. All the housings for the screws are intersected with the original cage and then combined into the porous cage as seen in the FIG. 19.
17. Material from all the holes of the cage are removed to form the final acetabular cage. The said formed acetabular cage is illustrated in the FIG. 20.

Finite element analysis Study comparison of Custom
Hip cage implant and Burch-Schneider cage implant

| Sr. | | | |
|---|---|---|---|
| No. | Description | Burch- Schneider Cage implant | Custom Hip cage implant |
| 1. | Type of | Paprosky 3B Defect | Paprosky 3B Defect |
| | defect in | | |
| | bone | | |
| 2. | Implant | FIG. 27a | FIG. 27b |
| | model | | |
| 3. | Material | Bone (cancellous): — | Bone (cancellous): — |
| | Properties | E-Modulus 300 MPa | E-Modulus 300 MPa |
| | | Implant (Titanium Ti6Al4V): — | Implant (Titanium Ti6Al4V): — |
| | | E-Modulus 110 GPa | E-Modulus 110 GPa |
| 4. | Boundary | All models were rigidly. | All models were rigidly. |
| | condition | fixed at the pubic symphysis and | fixed at the pubic symphysis and |
| | and value of | the sacroiliac joint of the hemi- | the sacroiliac joint of the hemi- |
| | force | pelvis and 1800N | pelvis and 2500N |
| | applied | Force applied in the cup of the | Force applied in the acetabular |
| | | Burch-Schneider cage implant. | cage 1 of the Custom Hip cage |
| | | | implant. |
| 5. | Von Mises | Maximum stress in implant: | Maximum stress in implant: |
| | Stress | 146.6 MPa | 72.6 MPa |
| | Values | Maximum stress in bone: | Maximum stress in bone: |
| | | 73.5 MPa | 80 MPa |

In the same type of Bone Defect The Burch-Schneider Cage implant shows more stress value of 146.6 MPa at 1800N force. It was observed that present invention Custom Hip cage implant 100 shows less stress value of 72.6 MPa at 2500N force.

Comparison of present invention with Burch-Shnider Reinforcement Hip cage from Zimmer:

| | Burch-Shnider Reinforcement Hip | Custom Hip Cage Implant from Jajal |
|---|---|---|
| Sr. No. | cage from Zimmer (FIG. 28a) | medical (FIG. 28b) |
| 1. | The shape of implant is standard and | The shape of implant is customized as |
| | can be bent during surgery to fit on | per patient's native bone, so it can be fit |
| | the bone. | easily without any work. For better |
| | | fitment given 0.5 mm clearance |
| | | between bone and implant interface. |
| 2. | The screws directions are not | The screws directions are customized |
| | customized, which results less bone | and designed as per maximum bone |
| | purchase. | purchase. |
| 3. | Implant does not have Locking | Implant has Locking screw compatible |
| | screws compatible holes, it only has | holes on the flange. Available screw |
| | non-locking 6.5 mm screw holes. | sizes are 6.5 mm, 5 mm and 3.5 mm. and |
| | | has non-locking screw holes for |
| | | 6.5 mm. |
| 4. | It does not have microporous | It has microporous structure with 65% |
| | structure for bone osteointegration. | to 72% porosity for bone |
| | | osteointegration. |
| 5. | It does not have provision to anchor | It has multiple grooves to anchor |
| | cement mantle. | cement mantle. These grooves are of |
| | | 1.2 mm wide. |

The preferred embodiment of the acetabular cage is manufactured by the process of 3D printing.

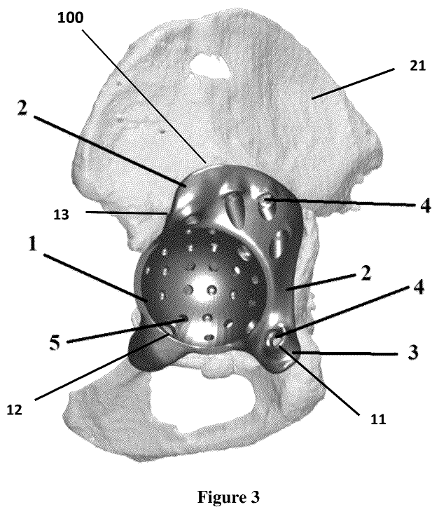

The FIG. 3 shows an illustration of the acetabular cage of the present invention implanted on a deformed hip bone.

In an embodiment of the present invention, the inner surface, i.e., the concave surface 1a is provided with surface roughness which enables the anchorage of the cement with the said surface. The plurality of bead cavities 5 further increase the said anchorage. The said design enables the usage of the said hip cage with any cemented implant liner.

In another embodiment of the present acetabular cage, the screw openings 4 are provided with threads which are designed to mate with locking screws.

The customized nature of the acetabular cage of the present invention, makes the said hip cage comparatively smaller than the hip implants of the prior art.

By completely customizing the design of the acetabular cage, the present invention minimizes the manual intervention required by the surgeon thus the time for the surgery is significantly reduced.

We claim:

1. A customized hip cage implant 100 comprising:
a customized acetabular cage 1 with integrated augment 2;
an outer side of acetabular cage 1 is a rear convex surface 1b (dome-shaped) surface having microporous structures 6, an inner surface, an opening acetabular cage 1;
one or more annular flange 3 extending from the periphery of the dome-shaped surface of acetabular cage 1;
a plurality of custom directed fixing screws comprises (a) locking Screws 8 (b) countersink dome screws 9 and (c) countersink compression screws 10 with hydroxyapatite stimulates the osteoinduction process and the structure of the surrounding bone tissue becomes significantly denser;
wherein the dome-shaped surface is a three-dimensional patient-specific surface designed to anatomically match and be received into an acetabulum of the patient.

2. The customized hip cage implant 100 as claimed in claim 1, wherein Locking Screws 8 is inserted into threaded holes 11 on integrated augment 2 passing through the cortical bone.

3. The customized hip cage implant 100 as claimed in claim 1, wherein countersink dome screws 9 is inserted into countersink dome holes 12 in acetabular cage 1 are the non-locking countersink acetabulum screws which are used to fix acetabular cage 1 with cortical and cancellous bone of the pelvis.

4. The customized hip cage implant 100 as claimed in claim 1, wherein countersink compression screws 10 is inserted into countersink compression holes 13 on integrated augment 2 are the non-locking countersink acetabulum screws which are used to fix acetabular cage 1 with bi-cortical bones on the iliac region to have compressive effect between the bone and implant surfaces.

5. The customized hip cage implant 100 as claimed in claim 1, wherein acetabular cage 1 with integrated augment 2 includes one or more annular flange 3 or dog ears 18 or elongated hook 17 having a patient-specific customized bone-engaging surface.

6. The customized hip cage implant 100 as claimed in claim 1, wherein the acetabular cage 1 can also optional include a straight elongated hook 17 between ischium and pubis bone.

7. The customized hip cage implant 100 as claimed in claim 1, wherein acetabular cage 1 with integrated augment 2 includes an annular flange 3 extending from the periphery of the dome-shaped surface of acetabular cage 1, the annular flange 3 having a bone-engaging surface positionable on a periacetabular area of the acetabulum of the patient, and wherein the plurality of resection guiding formations are defined through the annular flange 3.

8. The customized hip cage implant 100 as claimed in claim 1, wherein the annular flange 3 or dog ears 18 or elongated hook 17 and screws positions are customized for each case.

9. The customized hip cage implant 100 as claimed in claim 1, wherein the elongated annular flange 3 are arranged peripherally around the periphery of the dome-shaped surface.

10. The customized hip cage implant 100 as claimed in claim 1, wherein hip cage implant includes threaded hole at centre of acetabular cage 1 to fix impactor handle 14 during placement and positioning of hip cage implant 100.

11. The customized hip cage implant 100 as claimed in claim 1, wherein acetabular cage 1 arranged peripherally around with the dog ears 18 for custom locations for better bone purchase to resect the periacetabular area of the acetabulum 20 or the pelvis 21 for correction the hip dysplasia using a periacetabular resection procedure

12. The customized hip cage implant 100 as claimed in claim 1, acetabular cage 1 rear convex surface 1b is dome-shaped and has a complementary shape that matches the acetabulum 20 of the pelvis 21 of the patient for nesting therein in only one position; the annular flange 3 extends from a periphery of the rear convex surface 1b dome-shaped surface of the patient-specific acetabular cage 1.

13. The customized hip cage implant 100 as claimed in claim 1, wherein hip cage implant 100 can be patient-specific and designed during the pre-operative stage based on the three-dimensional image of the hip joint of the patient.

14. The customized hip cage implant 100 as claimed in claim 1, wherein hip cage implant 100 comprising acetabular cage 1 with integrated augment 2 the an outer surface is microporous structures 6.

15. The customized hip cage implant 100 as claimed in claim 1, wherein rear convex surface 1b of the acetabular cage 1 is provided with a plurality of ridges 7

16. The customized hip cage implant 100 as claimed in claim 1, wherein on the rear convex surface 1b of the acetabular cage 1 with integrated augment 2 is coated or covered with material promoting ingrowth, such a porous metal, including, porous titanium construct.

17. The customized hip cage implant 100 as claimed in claim 1, wherein the hip cage implant 100 are constructed by 3D printing, machining, casting, molding, stereolithography or layer deposition methods.

18. The customized hip cage implant 100 as claimed in claim 1, wherein the hip cage implant 100 are made of biocompatible metal alloys, titanium alloys (Ti-6A1-4V), or cobalt-chromium alloys (Co—Cr—Mo).

19. A customized hip cage implant 100 comprising: customized acetabular cage 1 with integrated augment 2
fix acetabular cage 1 with bi-cortical bones characterized by:
(a) locking Screws 8 inserted into threaded holes 11 on integrated augment 2 passing through the cortical bone, cancellous bone and then again cortical bone allowing bi-cortical purchase of bone to have stable fixation of the implant with bone and to avoid micromotion between bone and implant; (b) countersink dome screws 9 is inserted into countersink dome holes 12 in acetabular cage 1 are the non-locking countersink acetabulum screws which are used to fix acetabular cage 1 with cortical and cancellous bone of the pelvis; (c) countersink compression screws 10 is inserted into countersink compression holes 13 on integrated augment 2 are the non-locking countersink acetabulum screws which are used to fix acetabular cage 1 with bi-cortical bones on the iliac region to have compressive effect between the bone and implant surfaces.
(b) the process of fixing of the custom hip cage implant 100 with (a) Locking Screws (b) Countersink Dome Screws and (c) Countersink Compression Screws allows for the said hip cage implant to have integrated bone augment eliminating the requirement of loose pieces of bone augments thus reducing the risk of post operation implant failure;
wherein the length of these screws can be as high as 70 mm allowing the screw to hold the acetabular cage 1 to a pelvic region.
